SEQUENCE LISTING (2 sequences)

SEQ ID NO: 1 - DNA, 20 bp, artificial sequence
  sense primer for Shh
accgagggctgggacgaaga

SEQ ID NO: 2 - DNA, 18 bp, artificial sequence
  anti-sense primer for Shh
atttggcgccaccgagtt

(19) United States
(12) Patent Application Publication
(10) Pub. No.: US 2007/0059311 A1
(43) Pub. Date: Mar. 15, 2007

(54) Regulation of the hedgehog signaling pathway and uses thereof

(76) Inventor: Jingwu Xie, League City, TX (US)

Correspondence Address: Benjamin Aaron Adler;ADLER & ASSOCIATES, 8011 Candle Lane, Houston, TX, 77071, US

(21) Appl. No.: 11/519,531

(22) Filed: Sep. 12, 2006

Related U.S. Application Data
(60) Provisional application No. 60/716,329, filed on Sep. 12, 2005.

(51) Int. Cl. A61K 48/00; A61K 39/395; A61K 39/00; A61K 39/12; A61K 39/29; A61K 39/02

(52) U.S. Cl. 424/145.1; 424/185.1; 424/204.1; 424/228.1; 424/234.1; 514/44

(57) ABSTRACT

The present invention provides molecular mechanism leading to the development of cancers associated with hedgehog signaling pathway. These cancers may develop as a result of a chronic bacterial or a chronic viral infection. The present invention provides methods for early detection of such cancers. Additionally, it also provides measures that can be taken to prevent or treat such cancers.

### CROSS REFERENCE TO RELATED APPLICATION

This non-provisional application claims benefit of provisional application U.S. Ser. No. 60/716,329 filed on Sep. 12, 2005, now abandoned.

### FEDERAL FUNDING LEGEND

This invention was produced using finds obtained through a National Cancer Institute RO1CA94160. Consequently, the Federal government has certain rights in this invention.

### BACKGROUND OF THE INVENTION

1. Field of the Invention

The present invention relates to the field of cell signaling and treatment of cancer. More specifically, the present invention discloses methods of diagnosing and preventing or treating diseases such as cancer which may be caused due to chronic infection with viruses or bacteria and involve activation of the hedgehog signaling pathway.

2. Description of the Related Art

Hepatocellular carcinoma (HCC) is a malignancy of worldwide significance. It ranks fourth in cancer mortality worldwide and has become increasingly important in the United States. In the U.S., hepatocellular carcinoma represents a looming epidemic for which oncologists are unlargely unprepared. Despite the many treatment options, the prognosis of hepatocellular carcinoma remains dismal since a majority (70% to 85%) of the patients display an advanced or unresectable disease at the time of diagnosis. Furthermore, systemic chemotherapy is also quite ineffective in treatment of hepatocellular carcinoma. Additionally, although it is known that infection with a bacteria or a virus can lead to development of chronic disease, the molecular pathway leading to the development of the disease is not known. Examples of such chronic diseases include but are not limited to liver cancer, which is strongly associated with chronic infection with hepatitis C virus and stomach ulcers and stomach cancers that are associated with infection with Helicobacter pylori.

The hedgehog pathway is essential for embryonic development, tissue polarity and cell differentiation. It has been reported that the sonic hedgehog (Shh) gene is expressed in fetal liver but not expressed in the normal adult liver. Additionally, the role of hedgehog pathway in human cancers is well established through studies of basal cell nevus syndrome (BCNS), a rare hereditary disorder with a high risk of basal cell carcinomas. Activation of the hedgehog pathway is also observed in other cancers such as prostate cancer and gastrointestinal cancers. Additionally, targeted inhibition of the hedgehog pathway results in growth inhibition in cancer cell lines with activated hedgehog signaling.

Thus, the prior art in general is deficient in an understanding of the molecular pathway leading to the development of chronic diseases such as cancer and thus, lacks methods to diagnose and treat such diseases. The present invention fulfills this long-standing need and desire in the art.

### SUMMARY OF THE INVENTION

The present invention demonstrates that activation of the hedgehog signaling pathway is involved in the development of cancers such as liver cancer or in the development of cancer associated with viral or bacterial infection. It also provides markers that could be used to diagnose such cancers and drugs that could be used to treat such cancers.

The present invention detected expression of the sonic hedgehog gene, PTCH1 and Gli1 in 115 cases of hepatocellular carcinoma. Expression of the sonic hedgehog gene was high in half of the hepatocellular carcinomas. Consistent with this, the expression of hedgehog target genes PTCH1 and Gli1 was also high in over 40% of the hepatocellular carcinomas, thereby suggesting that the hedgehog pathway was frequently activated in hepatocellular carcinoma. Additionally, targeted inhibition of the hedgehog pathway in two such hepatocellular carcinoma cell lines resulted in apoptosis. Thus, the results presented herein indicated that activation of hedgehog signaling pathway was an important event during the development of subsets of hepatocellular carcinoma.

The present invention also demonstrated activation of the hedgehog signaling pathway in cancer cells such as liver cancer cells that were infected with the virus. Further, targeted inhibition of the hedgehog pathway in such cells resulted in apoptosis. H. pylori infects nearly half of the world population. The present invention demonstrated activation of hedgehog signaling pathway in cells such as gastric epithelial cells that were infected with H. pylori and induction of apoptosis upon targeted inhibition of the hedgehog pathway in such cells. Thus, the inhibition of hedgehog signaling pathway may be effective in eliminating the virally or bacterially infected cells, some of which may eventually lead to development of cancer.

In one embodiment of the present invention, there is a method of preventing or treating an infection caused by a virus or a bacteria in an individual. Such a method comprises administering a pharmacologically effective amount of an inhibitor that inhibits a gene encoding a protein or a protein that enables replication of said virus or said bacteria, a gene encoding a protein of hedgehog signaling pathway, hedgehog signaling pathway protein or combination thereof. Such administration causes apoptosis, inhibits proliferation of a virally or a bacterially infected cell or a combination thereof. Thus, preventing or treating the infection caused by the virus or the bacteria in the individual.

In another embodiment of the present invention, there is a method of treating or preventing a cancer caused by an activated hedgehog signaling pathway in an individual. Such a method comprises administering a pharmacologically effective amount of an inhibitor that inhibits viral or bacterial replication and/or inhibits activation of the hedgehog signaling pathway. Thus, preventing or treating the cancer caused by activated hedgehog signaling pathway in the individual.

In yet another embodiment of the present invention, there is a method of diagnosing an individual with a cancer associated with hedgehog signaling pathway. Such a method comprises obtaining a biological sample from the individual and determining expression of proteins associated with hedgehog signaling pathway in the biological sample, where detection of the protein in the sample indicates that the individual has the cancer associated with the hedgehog signaling pathway.

### BRIEF DESCRIPTION OF THE DRAWINGS

FIG. 1 is a diagrammatic representation of Hedgehog signaling pathway leading to the development of different cancers and the molecules in the pathway that are sensitive to inhibitors described herein.

FIGS. 2A-2D show detection of Gli1, PTCH1 expression in primary hepatocellular carcinoma. In situ hybridization detection of Gli1 (FIG. 2A) and PTCH1 (FIG. 2B) transcripts in hepatocellular carcinomas was performed. Positive signals (dark grey staining) were observed in tumor nests (indicated by arrows) and not in the surrounding stroma or in the liver tissues adjacent to the tumor (Normal). The sense probes did not give any positive signals confirming the specificity of the insitu hybridization expression of Gli1 and PTCH1 were confirmed by RT-PCR analysis (FIGS. 2C and 2D) in selected tumor samples. Data indicates values relative to 18S RNA and to a calibrator.

FIGS. 3A-3E show detection of shh expression in HCCs. In situ hybridization (FIG. 3A), real time RCR (FIG. 3B) and regular RT-PCR (FIG. 3C) were used to detect the Shh transcript. Shh transcript (dark grey signals in FIG. 3A) were observed in tumor nests (indicated by arrows) and not in the surrounding stroma or in the liver tissues adjacent to the tumor (Normal) and suggested that the tumor tissue was major source for Shh expression. The real time PCR results were confirmed by RT-PCR. Tumors with detectable Gli1 and PTCH1 had detectable Shh, suggesting a major role of Shh in the activation of hedgehog signaling pathway in HCCs. Additional real time PCR experiments showed a relatively high level of Gli1 (FIG. 3D), PTCH1 and Shh (FIG. 3E) in three HCC cell lines: Hep3B, Huh7 and PLC. Data indicates values relative to 18S RNA and relative to a calibrator.

FIGS. 4A-4F show expression of PTCH1 and β-catenin proteins in liver tissues. FIGS. 4A-4C show expression of PTCH1 protein as detected by immunohistostaining in subsets of liver cancer (positive in brown) and FIGS. 4D-4F show expression of β-catenin by immunohistostaining.

FIGS. 5A-5D show that activation of hedgehog signaling pathway in liver cancer cell lines growth of hepatocellular carcinoma cells. FIG. 5A identifies liver cancer cells having activated hedgehog signaling pathway using real-time PCR. A high level of Gli1 transcript indicated activation of the hedgehog pathway. Hep3B cell line had the highest level of Gli1 and Hep2G had the lowest level of Gli1, suggesting that Hep3B has activated hedgehog signaling whereas Hep2G did not. FIG. 5B shows that hedgehog signaling is required for the growth of liver cancer cells. In the presence of the SMO antagonist KAAD-cyclopamine (2 μM, as indicated as Cyc in the figure), Gli1 expression was decreased in Hep3B cells, but not in Hep2G cells. Trypan blue assay showed an increased level of cell death in Hep3B cells but not in Hep2G cells (FIG. 5B, top panel), suggesting apoptosis following cyclopamine treatment. TUNNEL assay revealed apoptosis (red) in Hep3B cells (FIG. 5B, bottom panel) but not in Hep2G cells. Cell growth of Huh7 cells (FIG. 5C) and HepG2 (FIG. 5D) were examined by MTT assay. Huh7 cells were inhibited by 2.5 μM KAAD-cyclopamine or 1 μg/ml Shh neutralizing antibodies. The inhibition was specific because addition of tomatidine, a non-specific compound for hedgehog signaling did not affect cell growth. In contrast, cell growth of HepG2 was not affected by KAAD-cyclopamine (2.5 μM) or Shh neutralizing antibodies (1 μg/ml).

FIG. 6 shows up-regulation of shh in HCV replicated cells. The Shh transcript level was measured in Huh7, Huh7-2-3(+), Huh7-2-3(−) and SIIA (control) cells by real-time PCR. The Shh transcript level was upregulated by active HCV replication as observed in Huh7-2-3(+) cells.

FIGS. 7A-7C show that activation of sonic hedgehog signaling pathway was associated with HCV replication. FIG. 7A shows activation of hedgehog target gene by HCV replication. The Gli1 transcript level was measured in Huh7, Huh7-2-3(+), Huh7-2-3(−) and SIIA (control) cells by real-time PCR. HCV replication in Huh7-2-3(+) cells induced elevated expression of Gli1 which was reduced following treatment with interferon as observed in Huh7-2-3(−) cells. FIG. 7B shows association of sonic hedgehog promoter activity with HCV replication. The promoter activity of Shh was higher in Huh7 cells than in HepG2 cells (Shh indicates Shh promoter activity and ctr indicates the vector control luciferase reporter construct). Following stable expression of HCV in Huh7 cells, there was a dramatic increase in shh promoter activity. This shh promoter activity was reduced when the HCV RNA replication was inhibited by interferon treatment. FIG. 7C shows induction of sonic hedgehog promoter activity by HCV infection. A 6-fold increase in the Shh promoter activity was observed after 12 hours following HCV infection, which was not observed 24 hr or 48 hrs after infection.

FIGS. 8A-8C show that cells with active HCV replication are sensitive to inhibition of hedgehog signaling pathway. Cells were treated with cyclopamine (cyc) at 2.5 μM for 24 hours. FIG. 8A shows apoptotic cells that were identified by abnormal nuclear morphology (indicated by arrow) when Huh7-2-3(+) cells were treated with cyc. FIG. 8B shows morphology of Huh7-2-3(−) cells when treated with cyc. FIG. 8C shows quantitative analysis of the TUNNEL staining of the cells (described herein) when treated with cyc. Over 15% of Huh7-2-3(+) cells underwent apoptosis following treatment with cyclopamine whereas only 6% of cell death was observed in Huh7-2-3(−) cells (p value<0.05 using Binomial Proportion analysis).

FIG. 9 shows activation of hedgehog signaling following infection with H. pylori bacteria. The expression of Gli1 was measured by real time PCR in HS754 control cells, cells infected with H. pylori and treated with cyclopamine or infected with H. pylori but not treated. Gli1 was induced as early as 8 hours following bacterial infection. However, in the presence of cyclopamine, the Gli1 transcript level was reduced.

FIGS. 10A-10B show that cells infected with H. pylori are sensitive to inhibition of hedgehog signaling. A fetal gastric epithelial cell line (HS754 cells) was infected with H. pylori for 24 hours and the cells were untreated or treated with 2.5 μM cyclopamine (KAAD-cyclopamine) for 24 hours or treated with 20 mM forskolin and 3-Isobutyl-1-methylxanthine (IBMX) for 5 hours followed by TUNNEL staining. FIG. 10A compares the morphology of the cells when untreated or treated with either cyclopamine or forskolin. FIG. 10B compares percentage of the TUNNEL positive cells when cells were untreated or infected with H. pylori alone, treated with cyclopamine alone, infected with H. pylori and treated with cyclopamine, treated with IBMX and forskolin or infected with H. pylori and treated with IBMX and forskolin.

FIGS. 11A-11B show that cells infected with H. pylori are sensitive to inhibition of hedgehog signaling. N87 gastric epithelial cells were infected with H. pylori for 24 hours. Cells were untreated or treated with 2.5 μM cyclopamine (KAAD-cyclopamine) for 24 hours or treated with 20 mM forskolin and IBMX for 5 hours followed by TUNNEL staining. FIG. 11A compares the morphology of the cells when untreated or treated with either cyclopamine or forskolin. FIG. 11B compares percentage of the TUNNEL positive cells when cells were untreated or infected with H. pylori alone, treated with cyclopamine alone, infected with H. pylori and treated with cyclopamine, treated with IBMX and forskolin or infected with H. pylori and treated with IBMX and forskolin.

FIGS. 12A-12B show that reduction of Hepatitis B virus (HBV) expression resulted in inhibition of hedgehog signaling in PLC/PRF/5 cells. FIG. 12A shows that inhibition of HBV replication by knocking down hepatitis B virus X protein (hbx) caused reduced cell growth in soft agar. FIG. 12B shows that siRNA treatment of PLC/PRF/5 cells resulted in 90% reduction of GLI1 transcript, thereby suggesting that HBV infection regulates hedgehog signaling.

### DETAILED DESCRIPTION OF THE INVENTION

Over one million new cases of liver cancers are reported worldwide each year, most of which are hepatocellular carcinomas. Most of the hepatocellular carcinoma patients, however, are diagnosed late and therefore cannot be treated effectively. Additionally, the lack of understanding of the molecular mechanism underlying the development of liver cancer further hampers the diagnosis and treatment of this disease.

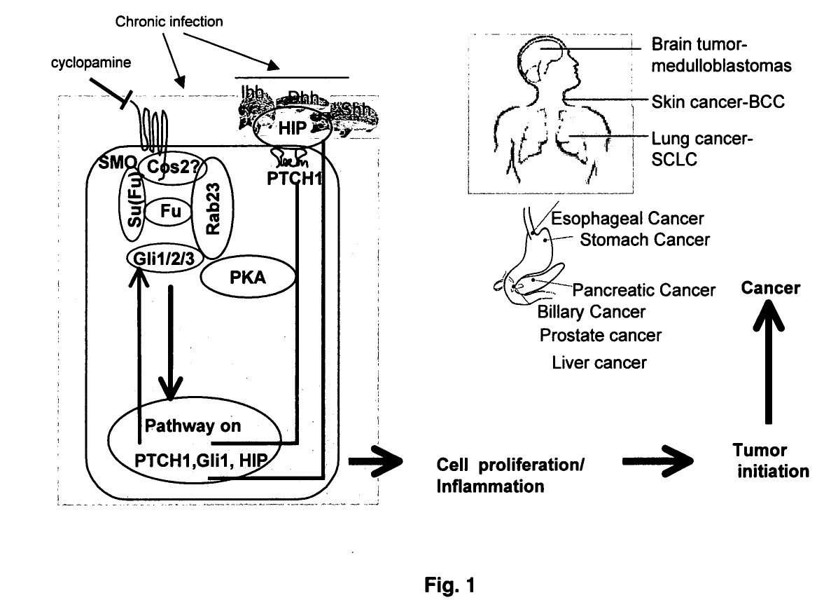

The present invention provides strong evidence regarding the activation of the hedgehog pathway in liver cancers. Additionally, it also provides evidence that this pathway is activated in cancers associated with viral (e.g. hepatitis C virus) and bacterial (e.g., H. pylori) infection. The mechanism underlying the development of such cancers is shown in FIG. 1. Chronic infection activates the hedgehog signaling pathway by inducing the expression of Shh, PTCH1 and Gli1 which induces cell proliferation or causes inflammation. Both of these lead to initiation of tumor development and finally to cancer. Examples of such cancers include but are not limited to brain tumors (medulloblastomas), skin cancer (basal cell carcinoma), lung cancer (small cell lung cancer), esophageal cancer, stomach cancer, pancreatic cancer, billiary cancer, prostate cancer and liver cancer. Thus, the present invention provides markers such as Shh, PTCH1 and GLI1 of activated hedgehog signaling pathway that may be useful in early diagnosis of such cancers. Several types of specimens can be used for assessing activation of hedgehog signaling pathway. Blood samples and fine needle aspirations (FNA) are the most convenient specimens for early diagnosis. The present invention also provides evidence for detection of activated hedgehog signaling pathway using serum of an individual and for detection of tumor cells in the fine needle aspirations specimens using antibodies specific for PTCH1 and Shh.

Furthermore, the present invention also investigated the association of the hedgehog signaling pathway with the wnt pathway in liver cancer. About 60% of liver cancers are reported to have activated β-catenin signaling. The results presented herein demonstrated that these two pathways were independent of each other in liver cancers. Additionally, these results also indicated that 40% of liver cancers had activated hedgehog signaling pathway. Since the tumors with activated hedgehog signaling pathway did not overlap with tumors with activated wnt signaling pathway, it is contemplated that the detection of both β-catenin signaling and hedgehog signaling will be more effective in the diagnosis of liver cancer.

Additionally, the present invention also provides evidence that targeted inhibition of hedgehog signaling may be effective in treatment of subsets of liver cancer. The present invention demonstrated that the SMO antagonist, KAAD-cyclopamine, specifically induced apoptosis in liver cancer cells with activated hedgehog signaling pathway. Since the hedgehog signaling pathway was not activated in Hep2G cells, these cells were not sensitive to KAAD-cyclopamine. Only 2 μM of KAAD-cyclopamine was sufficient to induce apoptosis within 12 hours, suggesting that this compound was very effective in killing liver cancers. Furthermore, the present invention also demonstrated that cyclopamine and a combination of forskolin and IBMX induced apoptosis in liver cancer cells that allowed Hepatitis C virus replication and in gastric epithelial cells infected with H. pylori, where the hedgehog signaling pathway was activated in both types of cells following viral or bacterial infection. Thus, it is contemplated that such cancers where the hedgehog signaling pathway is activated can be treated with either cyclopamine or forskolin and IBMX. The present invention further contemplates identifying herbal medicine capable of inhibiting hedgehog signaling to treat cells infected with H. pylori or hepatitis C virus or liver cancer cells.

The present invention is directed to a method of preventing or treating an infection caused by a virus or a bacteria in an individual, comprising: administering a pharmacologically effective amount of an inhibitor that inhibits a gene encoding a protein or a protein that enables replication of said virus or said bacteria, a gene encoding a protein of hedgehog signaling pathway, the hedgehog signaling pathway protein or combination thereof such that the administration causes apoptosis and inhibits proliferation of a virally or a bacterially infected cell or a combination thereof, thereby preventing or treating the infection caused by the virus or the bacteria in the individual. Generally, the inhibitor may inhibit the sonic hedgehog (Shh), Glioma-associated oncogene 1 (Gli1) or Patch1 (PTCH1) gene or the protein encoded by the gene. Examples of such inhibitors are not limited to but may include cyclopamine, forskolin and IBMX, SiRNA for Gli1 and Gli2, hedgehog neutralizing antibodies or recombinant hedgehog interacting protein (HIP). Furthermore, the virus may cause chronic viral infection and the examples of such virus is not limited to but may include hepatitis C virus, hepatitis B virus, Human papillomavirus (HPV), Epstein-Barr virus or Herpes simplex virus. Additionally, the bacteria may cause a chronic bacterial infection and the examples of such bacteria is not limited to but includes H. pylori, Bartonellae, or Agrobacterium. Further, the administration of the inhibitor described herein may treat a precancerous condition or prevent or treat cancer in the individual. The example of cancer treated by such a method may include but is not limited to liver cancer, stomach cancer, cervical cancer, lymphoma, oral squamous cell carcinoma or vascular tumor.

The present invention is further directed to a method of preventing or treating a cancer caused by activated hedgehog signaling pathway in an individual, comprising: administering a pharmacologically effective amount of an inhibitor that inhibits viral or bacterial replication and/or inhibits activation of the hedgehog signaling pathway, thereby preventing or treating the cancer caused by the activated hedgehog signaling pathway in the individual. Generally, the inhibition may cause apoptosis and/or inhibition of proliferation of virally or bacterially infected cell, may cause apoptosis and/or inhibition of proliferation of a cancer causing cell or a combination thereof. The activation of hedgehog signaling pathway may be inhibited by inhibiting a gene encoding a protein of the hedgehog signaling pathway or a hedgehog signaling protein. Specifically, the inhibition may inhibit Sonic hedgehog (Shh), Glioma-associated oncogene1 (Gli1) or Patch1 (PTCH1) gene or the protein encoded by the gene. Example of such an inhibitor is not limited to but may include cyclopamine, forskolin and IBMX, SiRNA for Gli1 and Gli2, hedgehog neutralizing antibodies or recombinant hedgehog interacting protein (HIP). The cancer that can be treated or prevented using the method described herein may be caused due to a chronic viral or a chronic bacterial infection. The examples of virus and bacteria infecting the cell are discussed supra. Example of a cancer that can be prevented or treated using this method is not limited to but may include liver cancer, stomach cancer, cervical cancer, lymphoma, oral squamous cell carcinoma or vascular tumor.

The present invention is still further directed to a method of diagnosing an individual with a cancer associated with hedgehog signaling pathway, comprising: obtaining a biological sample from the individual and determining expression of proteins associated with the hedgehog signaling pathway in the biological sample, wherein detection of the protein in the sample indicates that the individual has the cancer associated with hedgehog signaling pathway. This method may further comprise the step of concomitantly measuring the level of beta-catenin in the sample. Generally, the protein whose expression is determined may be Shh, PTCH1 or Gli1. Additionally, the biological sample used in this method may be a blood sample or a fine needle aspiration specimen. The individual who benefits from such diagnostic test may be the one likely to suffer from or the one suspected of having a cancer or a disease caused by activated hedgehog signaling pathway. Furthermore, the disease caused by activated hedgehog signaling pathway may be caused by a chronic viral or a chronic bacterial infection. Examples of the disease caused by the chronic viral infection is not limited to but may include hepatitis, cervical inflammation, or periodontitis and examples of the disease caused by the chronic bacterial infection is not limited to but may include stomach ulcers, gastritis, lymphadenitis or vertebral osteomyelitis. Additionally, examples of cancer are the same as discussed supra.

As used herein, the term, “a” or “an” may mean one or more. As used herein in the claim(s), when used in conjunction with the word “comprising”, the words “a” or “an” may mean one or more than one. As used herein “another” or “other” may mean at least a second or more of the same or different claim element or components thereof.

As used herein, the term “precancerous condition” may mean a condition or a tissue abnormality that is capable of becoming cancerous if left untreated.

The virally or bacterially infected cell or cancer cell may be contacted with the inhibitors described herein. As used herein, the term “contacting” refers to any suitable method of bringing the composition described herein into contact with a cell culture system that has been exposed to inflammatory stimuli. In vitro or ex vivo may be achieved by exposing the above-mentioned cell to the composition in a suitable medium.

The composition described herein can be administered either systemically or locally, by any method standard in the art, for example, subcutaneously, intravenously, parenterally, intraperitoneally, intradermally, intramuscularly, topically, enterally, rectally, nasally, buccally, vaginally or by inhalation spray, by drug pump or contained within transdermal patch or an implant. Dosage formulations of the composition described herein may comprise conventional non-toxic, physiologically or pharmaceutically acceptable carriers or vehicles suitable for the method of administration.

The composition described herein may be administered one or more times to achieve, maintain or improve upon a therapeutic effect. It is well within the skill of an artisan to determine dosage or whether a suitable dosage of either or both of the composition comprises a single administered dose or multiple administered doses. An appropriate dosage depends on the subject's health, the prevention or treatment of viral or bacterial infection, the treatment of cancer, the route of administration and the formulation used.

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

### EXAMPLE 1

Tissue Samples

A total of 115 specimens of hepatocellular carcinoma tissues were used. Of these, 14 specimens (pathology reports and H&E staining were reviewed to determine the nature of the disease and the tumor histology) were received from General Surgery of Shan Dong Qi Lu Hospital, Jinan, China and the remaining 101 HCC specimens were in tissue array from Sun Yat-Sen University. Forty-four matched normal liver tissues were used as controls. None of the patients had received chemotherapy or radiation therapy.

### EXAMPLE 2

In Situ Hybridization

In situ hybridization was performed according to the manufacturer's instructions (Boehringer Mannheim). Briefly, tissues were fixed with 4% paraformaldehyde in phosphate buffered saline and embedded in paraffin. Further, 6 μm thick tissue samples were mounted on Poly-L-Lysine slides. Samples were treated with proteinase K (20 μg/ml) at 37° C. for 15 min, refixed in 4% paraformaldehyde and hybridized overnight with a digoxigenin-labeled RNA probe (1 μg/ml). The hybridized RNA was detected by alkaline phosphatase-conjugated anti-digoxigenin (Roche Molecular Biochemicals) which catalyzed a color reaction of the substrate NBT/BCIP (Roche, Mannheim, Germany). Blue signal indicated positive hybridization. Tissues with no blue signal were regardes as negative. As negative controls, sense probes were used in the hybridization and no signals were observed. In situ hybridizations were repeated at least twice for each tissue sample with similar results.

### EXAMPLE 3

RNA Isolation and Quantitative RT-PCR

Total RNA of cells was extracted using a RNA extraction kit from Promega according to the manufacturer (Promega, Madison, Wis.) and qunatitative PCR analyses were performed according to the previously published procedure (Ma et al., 2005; Chi et al., 2006). Triplicate C_{T} values were analyzed in Microsoft Excel using the comparative C_{T} (ΔΔC_{T}) method as described by the manufacturer (Applied Biosystems, Foster City, Calif.). The amount of target (2^{-ΔΔCT}) was obtained by normalization to an endogenous reference (18S RNA) and relative to a calibrator. The following primers were used for RT-PCR of Shh: forward primer-5′-ACCGAGGGCTGGGACGAAGA-3′; reverse primer-5′-ATTTGGCGCCACCGAGTT-3′ (SEQ ID Nos: 1 and 2).

### EXAMPLE 4

Cell Culture, Transfection and Drug Treatment

HCC cell cell lines (Hep3B, HepG2, HCC36, PLC/PRF/5 (identified as PLC) and Huh 7) were provided by Chiaho Shih, Tien Ko and Kui Li at UTMB. All cells were cultured in Dulbeco-modified essential medium (DMEM) with 10% FBS and antibiotics. Cells were treated with 2 μM KAAD-cyclopamine, a specific antagonist of smoothened (SMO) (dissolved in DMSO as 5 mM stock solution, Car# K171000 from Toronto Research chemicals, Canada) in 0.5% FBS in DMEM for indicated time. The toxicity assay with KAAD-cyclopamine in GI cancer cells had demonstrated that 10 μM of KAAD-cyclopamine could cause non-specific toxicity (Ma et al., 2006). In fact, 5 or 10 μM KAAD-cyclopamine was quite toxic to cells regardless of hedgehog signaling Tomatidine (2 μM in 0.5% FBS DMEM, Sigma Cat#T2909), a structurally similar compound with non-specific inhibition on hedgehog signaling pathway was used as a negative control. In addition, the specific inhibition of hedgehog signaling in HCC cells was achieved by addition of Shh neutralizing antibodies (1 μg/ml in 0.5% FBS DMEM, Car# SC-9024, Santa Cruz Biotechnology, Santa Cruz, Calif.). Most cell lines were treated with KAAD-cyclopamine (2.5 μM) or Shh antibodies (1 μg/ml) in 0.5% FBS DMEM medium. Transient transfection of Gli1 in HCC cells was performed using LipofectAmine according to the manufacturer's recommendation (Plasmid-LipofectAmine=1:2.5). Cells with ectopic expression of Gli1 were subjected to drug treatment and to TUNEL (terminal deoxynucleotidyl transferase-mediated dUTP nick end labeling) assay.

### EXAMPLE 5

Cell Viability and TUNEL Assays

For cell viability analysis, two methods were used: trypan Blue analysis and MTT assay. Trypan Blue analysis was performed to manufacturer's instruction (Invitrogen, CA). The percentage of trypan blue positive cell (dead cells) were calculated under a microscope and triplicates of samples for each treatment were used in a 96-well format. Twenty microliters of MTT (10 mg/ml in PBS) was added to each well (containing 100 μl cultured medium, 0.5% FBS DMEM). Three hours later, the medium was aspirated and 100 μl of a mixture of isopropanol and DMSO (9:1) was added into each well. Thirty minutes later, absorbance was measured at 570 nM using a microplate reader from Molecular Devices Co., Sunnyvale, Calif. BrdU labeling was for 1 h and immunofluorescent staining of BrdU was performed as described previously (Xie et al., 2001).

TUNEL assay was performed using a kit from Roche Biochemicals according to published procedure (Athar et al., 2004). Briefly, cells were fixed with 4% paraformaldehyde at room temperature for 1 h and permeated with 0.1% Triton X-100, 0.1% sodium citrate (freshly prepared) on ice for 2 min. After washing with PBS, each sample was incubated with 50 μl of TUNEL reaction mixture at 37° C. for 30 min. TUNEL labeled solution (without enzyme) was used as a negative control. TUNEL positive cells were counted under a fluorescent microscope. The counting was repeated three times and the percentage from each counting was calculated.

### EXAMPLE 6

Immunohistochemistry

The paraffin-embedded tissue sections that were cut into 6 μM thick sections and embedded on Poly-L-lysine coated slides were deparaffinized. Slides from each case were exposed to 1:200 dilution of affinity purified anti-PTCH or anti-sonic hedgehog primary antibody diluted in PBS (Santa Cruz, USA) for 2 hours at room temperature. Detection was carried out with a 1:100 dilution of biotinylated anti-rabbit IgG secondary antibody solution (Boshide, Calif.) for 30 min at 37° C. Sections were then incubated with avidin-biotin immunoperoxidase (Boshide, Calif.). The location sites of peroxidase were visualized with DAB. Hematoxylin was used for counterstaining. The sections were thoroughly washed with PBS between steps. Negative controls (absence of primary antibody) were run for each slide.

### EXAMPLE 7

Statistical Analysis

Statistical Analysis was performed by Binomial proportions analysis. The association of mRNA transcript with the various clinicopathological parameters was also estimated, a p value<0.05 was considered to be significant.

### EXAMPLE 8

Expression of PTCH1 and Gli1 in Primary HCC

The following three methods were used to examine activation of hedgehog signaling in hepatocellular carcinoma: in situ hybridization, immunohistochemistry and real time PCR and the results are summarized in Table 1. Since PTCH1 and Gli1 are known to be the target genes of the hedgehog pathway, increased levels of these transcripts indicated activation of the hedgehog signaling pathway.

TABLE 1
Detection of Shh, PTCH1 and GU1 expression in HCC and in adjacent liver tissue by in situ hybridization

| | | | | Hedgehog pathway activation, | | | | | | |
|---|---|---|---|---|---|---|---|---|---|---|
| | Shh | | | PTCH1 | | GU1 | | Pathway activation | | |
| | pos | neg | P-value | pos | neg | pos | neg | pos | neg | p-value |
| HCC | 64/108 | 44/108 | <0.01* | 60/107 | 47/107 | 79/110 | 31/110 | 57/98 | 47/98 | <0.01* |
| Adjacent tissues | 5/41 | 36/41 | | 18/43 | 25/43 | 15/44 | 29/44 | 9/43 | 34/43 | |
| Tumor size | | | | | | | | | | |
| Small (<3 cm) | 16/31 | 15/31 | 0.316 | 17/31 | 14/31 | 25/32 | 7/32 | 16/31 | 15/31 | 0.896 |
| Large (>3 cm) | 46/74 | 28/74 | | 42/74 | 32/74 | 52/75 | 23/75 | 35/66 | 31/66 | |
| Tumor differentiation | | | | | | | | | | |
| Well | 34/52 | 18/52 | 0.107 | 30/51 | 21/51 | 43/52 | 9/52 | 29/51 | 22/51 | 0.264 |
| Mod-poor | 20/41 | 21/41 | | 22/41 | 19/41 | 32/43 | 11/43 | 19/42 | 23/42 | |
| Sex | | | | | | | | | | |
| Male | 47/81 | 34/81 | 0.651 | 43/81 | 38/81 | 58/83 | 25/83 | 35/72 | 37/72 | 0.258 |
| Female | 17/27 | 10/27 | | 17/26 | 9/26 | 21/27 | 6/27 | 16/26 | 10/26 | |
| Hepatocirrhosis | | | | | | | | | | |
| + | 14/19 | 5/19 | 0.163 | 14/20 | 6/20 | 14/20 | 6/20 | 11/17 | 6/17 | 0.251 |
| − | 49/87 | 38/87 | | 43/83 | 40/83 | 63/87 | 24/87 | 39/79 | 40/79 | |

Statistical analysis was performed by Binomial proportions analysis. A P value <0.05 was considered statistically significant. The association of mRNA transcript expression with various clinicopathological parameters was also analyzed.
Statistically significant differences are indicated by asterisk (*).
Pos, positive signal; neg, negative signal; well-differentiated tumors; mod-poor, moderately to poorly differentiated tumors.
Elevated expression of at least two hedgehog target genes was regarded as being positive (pos) in activation of the hedgehog pathway, whereas elevated expression of one hedgehog target gene was regarded as being negative (neg) in hedgehog signaling activation.

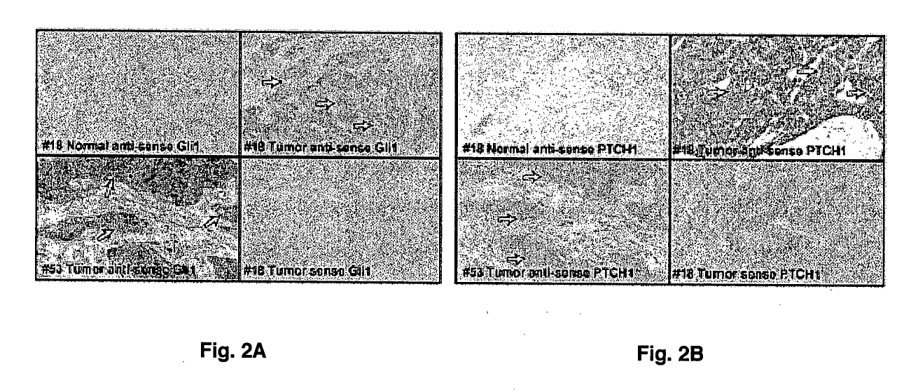

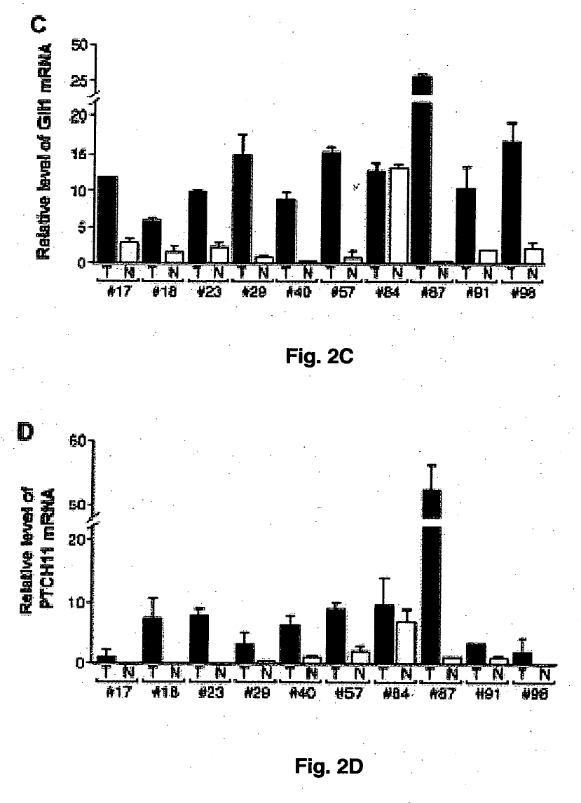

The in situ hybridization detected PTCH1 expression in 60 of 107 (56%) of tumor specimens (FIGS. 2A-B). In contrast, all normal matched liver tissues did not have detectable level of PTCH1. The sense probes gave no detectable signals (not shown), thereby indicating specificity of in situ hybridization. Positive staining was observed in the tumor nests and not in the stroma (FIGS. 2A-B). It was also observed that the signal intensity of the Gli1 transcript was generally stronger compared to the PTCH1 transcript. Around 70% (79/110) of HCC specimens were positive for expression of Gli1 transcript. Additionally, 51 tumors out of 98 informative HCCs (52%) showed detectable expression of both Gli1 and PTCH1 (Table 1), which suggested that activated hedgehog signaling occurred more frequently in HCC than in the adjacent liver tissue (Table 1). However, there were several cases where either PTCH1 alone or Gli1 alone was expressed, suggesting other regulatory mechanisms for Gli1 and PTCH1 expression. Nevertheless, the data presented herein indicated that the hedgehog signaling pathway was frequently activated in HCCs. Further analysis of the data did not reveal association of activation of the hedgehog signaling pathway with tumor size or differentiation (Table 1). Tumors with hepatocirrhosis were not significantly different from tumors without hepatocirrhosis in the expression of Gli1 or PTCH1 (Table 1).

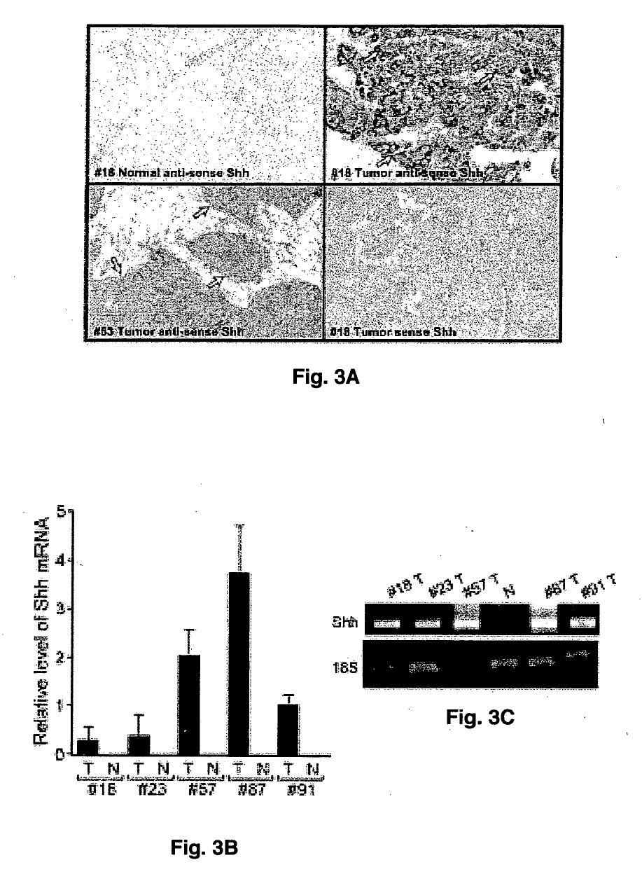

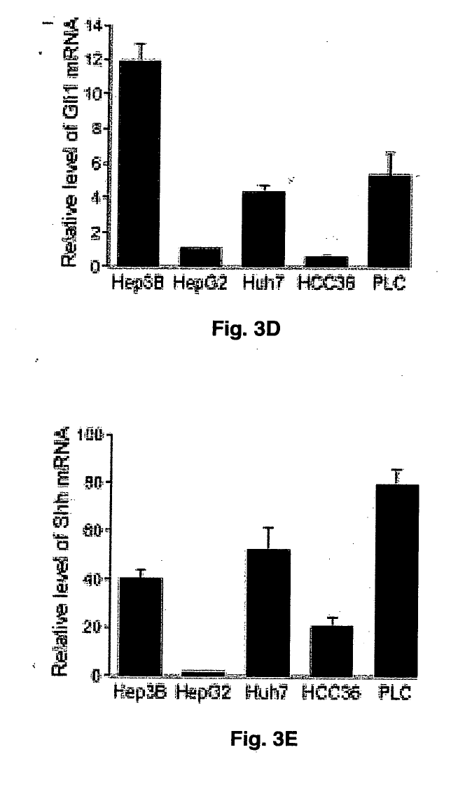

The results of in situ hybridization were further confirmed by performing real-time PCR in several tumor specimens in which 70% of the tissue mass actually tumor tissue. Consistent with the in situ hybridization, expression of Gli1 and PTCH1 were detected in tumor and not in the adjacent liver tissue in most cases. The levels of expression of Gli1 and PTCH1 in several tumors were over 3- to 30-fold higher than those in the controls (FIGS. 3A-B). The real time PCR analyses further confirmed that activation of the hedgehog pathway was a common event in HCC.

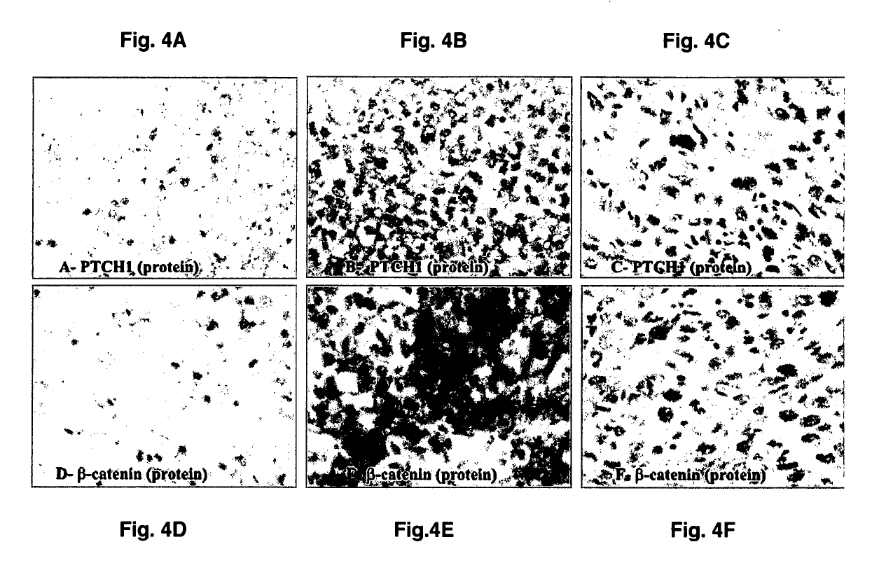

Additionally, PTCH1 protein was also detected by immunohistochemistry (FIG. 4A-C). It was observed that tumors that were positive for PTCH1 transcript were also positive for PTCH1 protein expression. Thus, the results of in situ hybridization, real-time PCR and immunohistochemical analyses indicated activation of the hedgehog pathway as a common event in liver cancers.

### EXAMPLE 9

Expression of Shh in HCCs

To investigate if sonic hedgehog protein expression was associated with activation of hedgehog signaling in hepatocellular carcinoma, sonic hedgehog protein expression was first detected by in situ hybridization. Sonic hedgehog protein expression was undetectable in normal liver tissue. However, expression of sonic hedgehog protein transcripts were detected in 64 of the 108 hepatocellular carcinoma specimens but not in majority of liver tissues adjacent to the tumor (Table 1). The Shh transcript was detectable specifically in the tumor nests and not in the stroma (dark grey signals in FIG. 3A), suggesting that the cancer cells were the source for sonic hedgehog protein expression. Expression of sonic hedgehog protein was further confirmed by performing immunohistochemistry using sonic hedgehog protein specific antibodies (not shown). No distinct difference was observed when various clinicopathological parameters were considered (table 1). Almost all tumors with detectable Gli1 and PTCH1 expression had detectable Shh transcripts (FIGS. 2, 3; Table 1). Shh expression in the tumor was further confirmed by real time PCR and regular RT-PCR (FIGS. 3B and 3C). Thus, it appeared that Shh induction may be the trigger for activated hedgehog signaling in HCCs. In support of this, expression of Shh was detected in all three HCC cell lines with detectable transcript of Gli1 (FIGS. 3D and 3E).

### EXAMPLE 10

Activation of Hedgehog Signaling does not Associate with the wnt Pathway

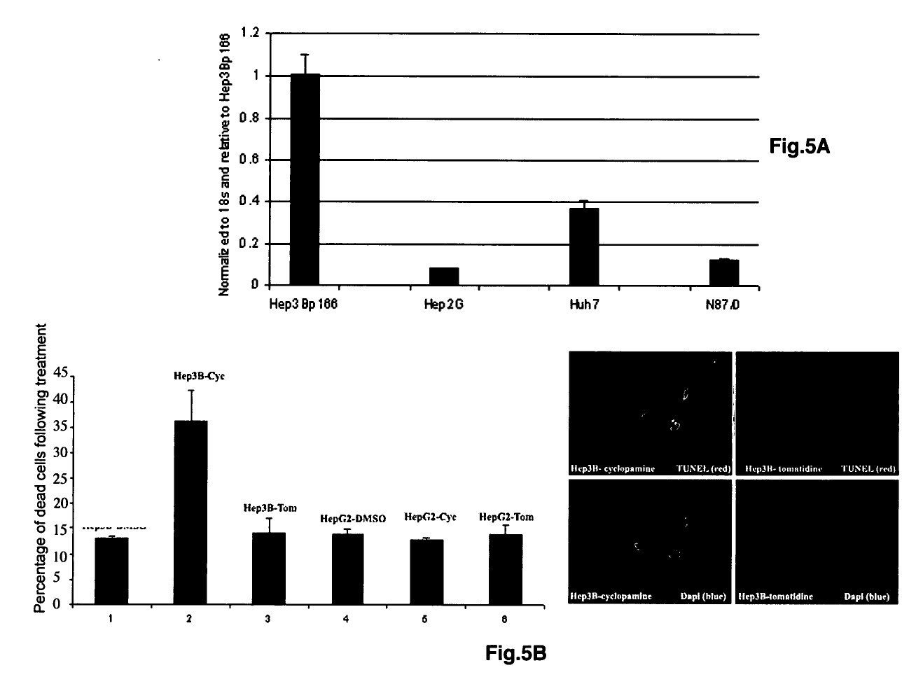

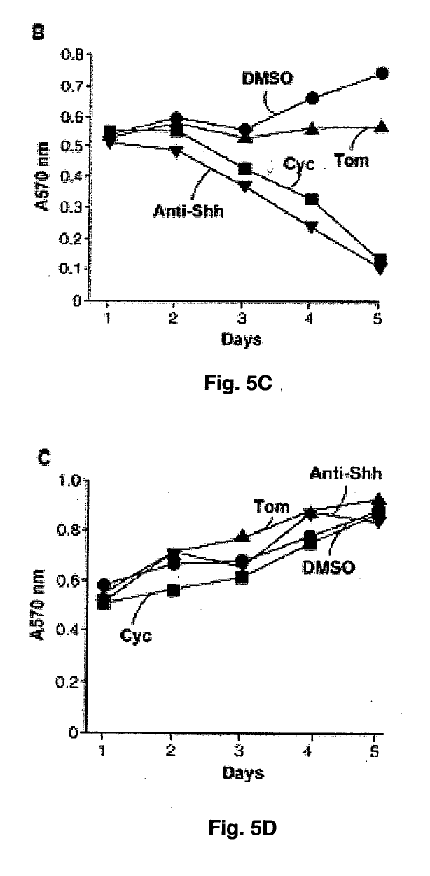

The wnt signaling pathway is activated in subsets of liver cancer via either mutation of β-catenin or inactivation of axin and the activated wnt signaling pathway is detectable by cytoplasmic or nuclear accumulation of β-catenin. Hence, the association of activation of hedgehog signaling with the wnt signaling was examined in the liver cancers by examining the localization of β-catenin protein in tumors with activated hedgehog signaling. Only 1 in 20 tumors with activated hedgehog signaling had nuclear β-catenin, an indicator of activated wnt signaling (FIG. 4E). This observation suggested that these two pathways were independent of each other during development of hepatocellular carcinoma, which was further confirmed in liver cancer cell line Hep2G (FIG. 5B).

### EXAMPLE 11

Targeted Inhibition of Hedgehog Signaling in Liver Cancer Cells

Although the hedgehog signaling pathway was activated in the liver cancer specimens, whether the activation of this pathway was required for liver cancer development was examined. It is known that if activation of hedgehog signaling pathway was required for liver cancer development, then the hepatocellular carcinoma cells should be susceptible to treatment with the SMO antagonist, KAAD-cyclopamine.

When the hepatocellular carcinoma cell lines were screened for activation of hedgehog signaling pathway, it was observed that this pathway was activated in Hep3B and Huh7 cells but not in Hep2G cells (FIG. 5A). The Hep2G cells have activated β-catenin signaling due to deletion of exon 3. Addition of KAAD-cyclopamine (2 μM) greatly decreased the levels of Gli1 and PTCH1 transcripts in Hep3B cells (not shown), indicating inhibition of the hedgehog pathway by KAAD-cyclopamine in these cells. The closely related compound tomatidine, which does not affect SMO signaling was used as a negative control and demonstrated little discemible effect on these target genes.

Additionally, it was observed that the cell growth of Hep3B cells but not Hep2G cells was inhibited by KAAD-cyclopamine (2 μM, not shown). Furthermore, an increased number of apoptotic cells were detected in Hep3B cells following treatment with cyclopamine whereas tomatidine did not induce apoptosis in both the cell lines (FIG. 5B). In distinct contrast, Hep2G cells, which do not have activated hedgehog signaling pathway were not affected by KAAD-cyclopamine treatment and showed no detectable apoptosis (FIG. 5B). Thus, the TUNNEL assay confirmed the data derived from the trypan blue analysis (FIG. 5B). The results obtained using Huh7 cell line, which is another cell line with activated hedgehog signaling pathway were similar to the results obtained using Hep3B cell line. The percentage of apoptotic cells varied from cell line to cell line with PLC being the most sensitive cell line.

Furthermore, cell growth of Huh7 (FIG. 5C) and HepG2 (FIG. 5D) cell lines was also examined by MTT assay. An inhibition of cell growth of Huh7 cell but not HepG2 cells was observed due to KAAD-cyclopamine treatment. The specificity of hedgehog signaling inhibition was further demonstrates using Shh neutralizing antibodies (FIGS. 5C and 5D). Addition of Shh antibodies at a concentration of 1 μg/ml reduced cell growth of Huh7 cells but had no effect on HepG2 cells (FIGS. 5C and 5D). Further analysis showed that BrdU incorporation was also reduced after treatment with KAAD-cyclopamine in Huh7 cells. Overall, these results indicated that HCC cells with activated hedgehog signaling are sensitive to targeted inhibition of hedgehog signaling pathway whereas other HCC cells are resistant to these treatments.

Since KAAD-cyclopamined and Shh antibodies only affect signaling upstream of SMO, it is hypothesized that cells with ectopic expression of the downstream effector Gli1 may prevent KAAD-cyclopamine-mediated apoptosis is these treatments are specific to the hedgehog pathway. It was observed that in Huh 7 cells that transiently expressed Gli1 under the control of CMV promoter, were negative for TUNEL when treated with KAAD-cyclopamine. These cells were also resistant to treatment with Shh antibodies. This suggests that downregulation of Gli1 may be important for targeted inhibition of hedgehog signaling pathway and may mediate apoptosis in HCC cells.

Taken together, the results discussed thus far demonstrate that activation of the hedgehog signaling pathway was quite common in liver cancers and that expression of Shh and Gli1 and PTCH1 was more frequent in tumor than in the adjacent liver tissue. This activation of hedgehog signaling pathway was not associated with other clinicopathological parameters of the tumor. HCC cells with activated tumor cells were sensitive to targeted inhibition of hedgehog signaling. Thus, these results support the hypothesis that activation of the hedgehog pathway was an important event in the development of HCC.

### EXAMPLE 12

Expression of Shh and Gli1 in Cells

The activation of hedgehog signaling pathway in hepatocellular carcinoma infected with virus or bacteria was also examined. Huh7 cells are hepatocellular carcinoma cells that can support HCV replication (Scholle F. et al., 2004. Huh7-2-3(+) are Huh7 cells that allow active HCV replication and Huh7-2-3(−) cells are derived from Huh7-2-3(+) cells following interferon treatment for two weeks to prevent virus replication. SIIA are gastric cancer cells with hedgehog signaling activation and used as control.

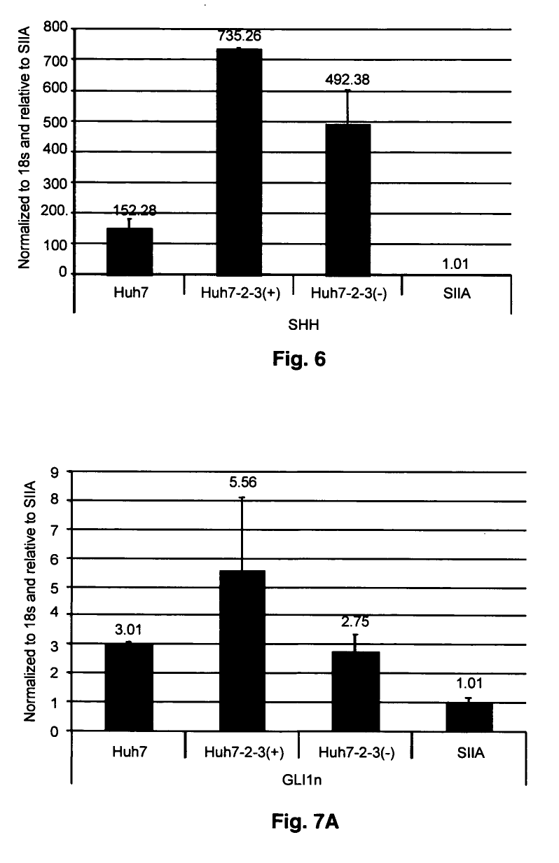

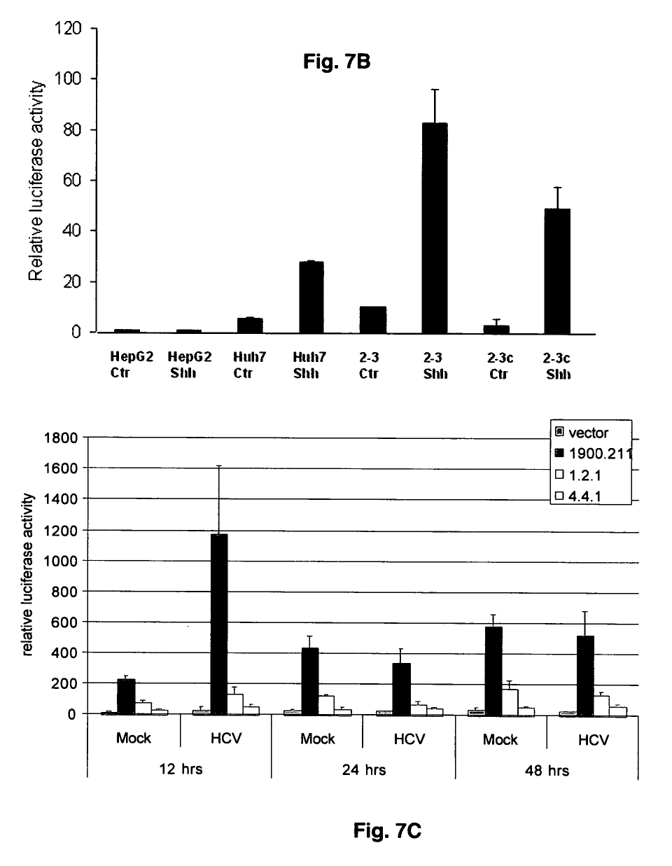

The present invention examined the expression of Shh and Gli1 transcript expression in the Huh7, Huh7-2-3(+), Huh7-2-3(−) and SIIA cells. It was observed that expression of Shh transcript (FIG. 6) and Gli1 transcript (FIG. 7A) were induced in Huh7-2-3(+) cells that allow HCV to replicate. In distinct contrast, Huh7-2-3(−) cells that do not allow the virus to replicate showed lower levels of expression of Shh and Gli1 transcript compared to the Huh7-2-3(+) cells. Additionally, the parent cells (Huh7) and the control cells (SIIA) showed low levels of expression of Shh and Gli1 transcripts.

The results obtained herein indicated that the Shh transcript expression could be up-regulated by active HCV replication. This observation was supported by the finding in gastrointestinal (GI) cancers where it has been shown that elevated Shh expression is the major reason for activation of hedgehog signaling pathway in GI cancers. Additionally, with regards to Gli1 transcript expression, the results obtained herein indicated that HCV replication in Huh7-2-3(+) cells induced elevated expression of Gli1 which was reduced following treatment with interferon as observed in Huh7-2-3(−) cells. Overall, these results demonstrated that active HCV replication induced activation of hedgehog signaling pathway.

Furthermore, the present invention also examined whether HCV RNA replication was a major trigger for activated hedgehog signaling in Hepatocellular carcinoma. In order to do so, cell lines with or without HCV RNA replication for the sonic hedgehog (shh) promoter activity were compared. The promoter activity of SHH was higher in Huh7 cells than in HepG2 cells. Following stable expression of HCV in Huh7 cells, shh promoter activity was dramatically increased. However, the shh promoter activity was reduced when the HCV RNA replication was inhibited by interferon treatment as shown in 2-3c (FIG. 7B). This data demonstrated that HCV RNA replication regulated Shh promoter activity.

Additionally, the present invention also measured the Shh promoter activity following infection of HCV in Huh7 cells at different times. 1900.211 is the construct covering the full length promoter of Shh whereas 1.2.1 and 4.4.1 have only part of the Shh promoter. A 6-fold increase in the Shh promoter activity was observed 12 hours following HCV infection. However, this effect was not observed 24 hr or 48 hrs after infection (FIG. 7C). Thus, the data presented herein demonstrated that HCV infection significantly induced Shh promoter activity and the persistent infection with HCV elevated the hedgehog signaling pathway. Thus, the data presented herein indicated that the hedgehog signaling pathway was an important pathway in response to chronic infection.

### EXAMPLE 13

Targeted Inhibition of Hedgehog Signaling Pathway in HCC Cells that Allow Replication of HCV

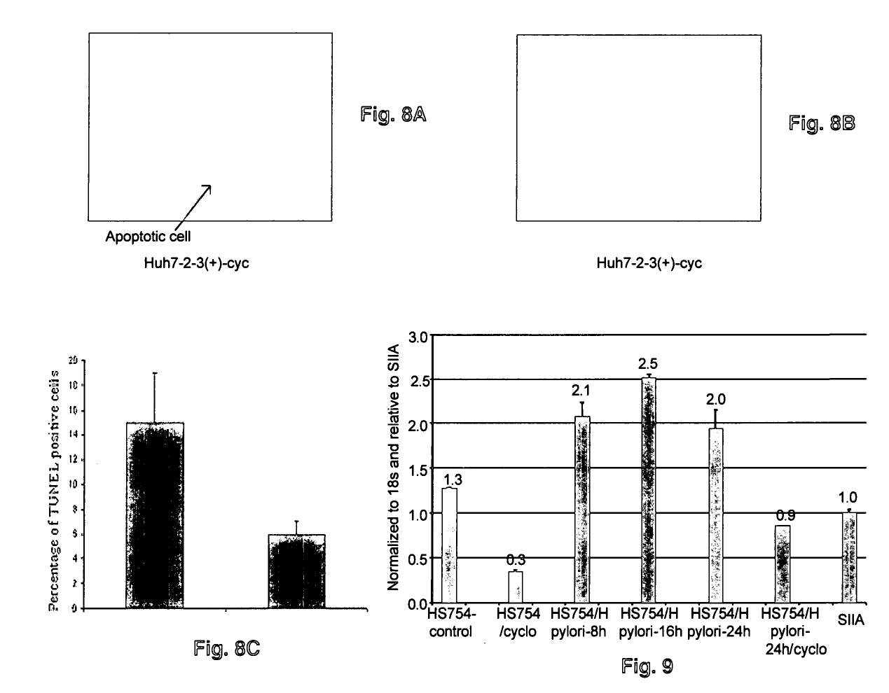

Since hedgehog signaling pathway was activated in hepatocellular carcinoma cells that allowed the HCV virus to replicate, the sensitivity of these cells to the inhibitors of hedgehog signaling pathway was examined. In order to accomplish this, Huh7-2-3(+) and Huh7-2-3(−) cells were treated with 2.5 μM cyclopamine (cyc) for 24 hours and the morphology of the cells examined to identify cells with abnormal nuclear morphology that represented apoptotic cells. Apoptotic cells were detected in Huh7-2-3(+) cells that were treated with cyc (FIG. 8A) whereas the nuclear morphology was intact in Huh7-2-3(−) cells that were treated with cyc (FIG. 8B). Further, to confirm these results and to quantitate the number of apoptotic cells, a TUNNEL assay was performed as previously described. The TUNNEL assay confirmed the results of the morphological examination of the cells. Additionally, over 600 cells were counted to obtain the percentage of TUNNEL positive cells in the two different cells. It was observed that over 15% of the cells underwent apoptosis in Huh7-2-3(+) cells following cyclopamine treatment compared to only 6% in Huh7-2-3(−) cells (FIG. 8C). This analysis was statistically significant since the p value obtained using Binomial Proportion was<0.05. Thus, the results obtained herein demonstrated that cells with active HCV replication were more sensitive to drugs inhibiting hedgehog signaling pathway.

### EXAMPLE 14

Expression of Shh and Gli Transcripts in Fetal Gastric Epithelial Cells Infected with H. pylori

The activation of hedgehog signaling pathway was also investigated in fetal gastric epithelial cells subsequent to infection with H. pylori. HS754 cells obtained from ATCC were infected with H. pylori at the bacteria:cell ratio of 30:1 for 24 hours. Expression of Gli1 was measured by real time PCR as previously described (Sheng, T et al., 2004) at different time points after infection such as 8 hours, 16 hours, 24 hours. In addition to measuring Gli1 expression in the cells that were infected with the bacteria, the expression was also measured in HS754 cells without such infection and no treatment or treatment with cyclopamine and SIIA (control) cells. It was observed that Gli1 was induced following bacterial infection as early as 8 hours. However, in the presence of cyclopamine the Gli1 transcript level was reduced, indicating that the mechanism of activation of hedgehog signaling pathway was through a molecule at SMO or in the upstream (FIG. 9).

Although the Gli1 transcript level was induced after bacterial infection in HS754 cells, the Shh transcript level was not induced subsequent to H. pylori infection. Similar results were obtained with another gastric cell line, N87 (immortalized gastric epithelial cells). Thus, the results obtained herein demonstrated that chronic infection of H. pylori was sufficient to activate hedgehog signaling pathway in fetal gastric epithelial cells which resemble normal human gastric epithelial cells.

### EXAMPLE 15

Targeted Inhibition of Hedgehog Signaling Pathway in Gastric Epithelial Cells Infected with H. pylori

Since the hedgehog signaling pathway was activated in the gastric epithelial cells following bacterial infection, the sensitivity of these cells to inhibitors of hedgehog signaling pathway was also examined.

HS754 cells infected with H. pylori for 24 hours were either untreated or treated with 2.5 μM cyclopamine (KAAD-cyclopamine) for 24 hours or a combination of ibmx and 20 mM forskolin for 5 hours. Forskolin was used in combination with IBMX since in the presence of IBMX, it increases cellular cAMP level, leading to elevated PKA activity and inhibition of hedgehog signaling. In addition to these cells, the effect of inhibitors was also examined in HS754 cells with no infection and no treatment or treatment with cyclopamine or ibmx and forskolin. TUNNEL assay was performed as to detect the apoptotic cells as well as to quantitate the number of apoptotic cells.

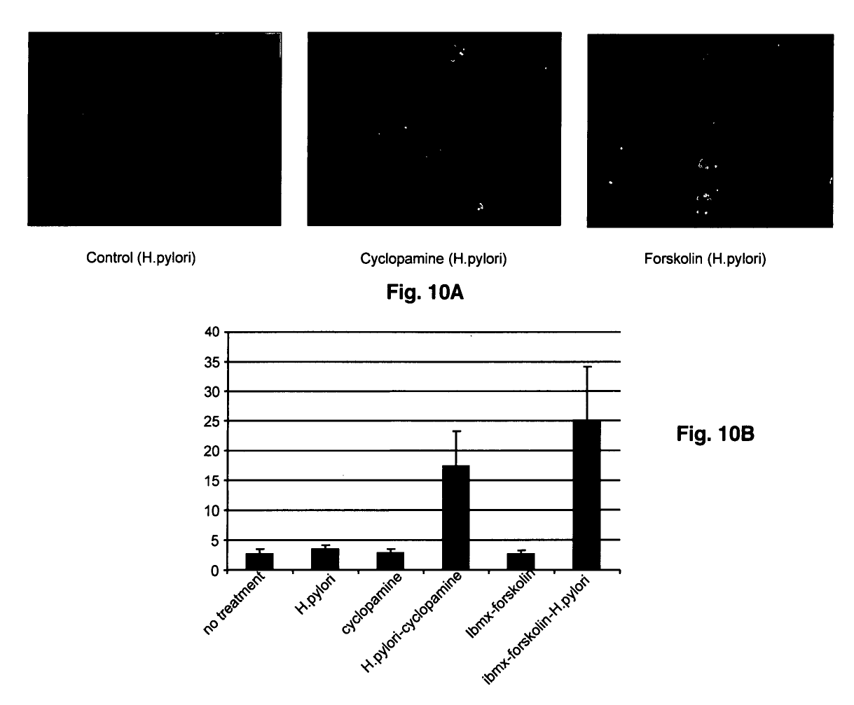

TUNNEL assay detected apoptotic cells in the HS754 cells that were treated with either cyclopamine or ibmx and forskolin (FIG. 10A) compared to HS754 cells that were infected with H. pylori but not treated. Quantitative analysis of the TUNNEL assay where over 600 cells were counted to obtain the percentage of apoptotic cells followed by statistical analysis using Binomial Proportion showed a statistically significant increase in the percentage of TUNNEL positive cells following treatment with either inhibitors following infection with the bacteria (FIG. 10B).

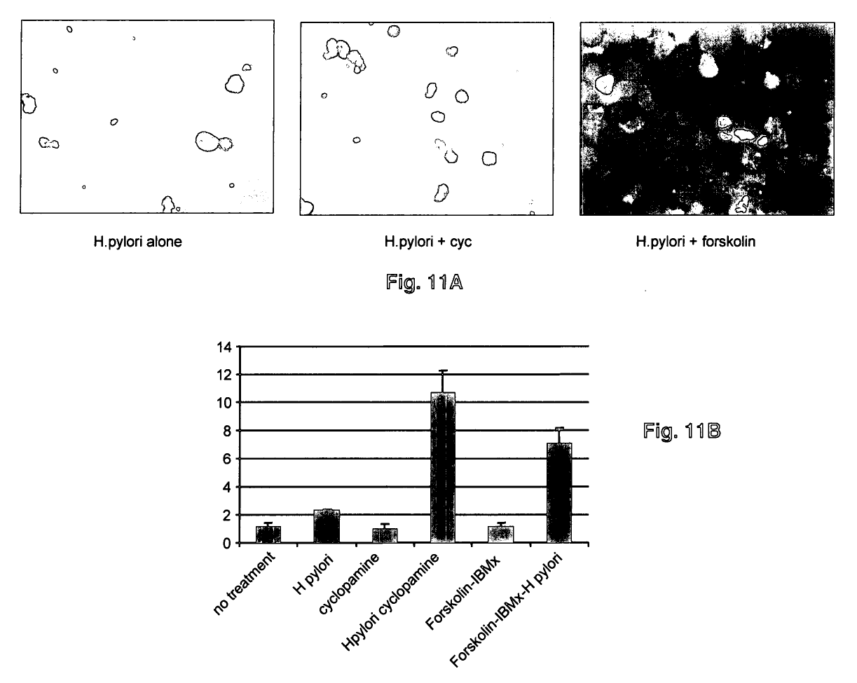

Additionally, TUNNEL assay was also performed to determine presence of apoptotic cells in N87 cells infected with H. pylori for 24 hours and either untreated or treated with 2.5 μM cyclopamine for 24 hours or 20 mM forskolin and IBMX. As discussed supra, the effect of inhibitors was also examined in N87 cells with no infection and no treatment or treatment with cyclopamine or ibmx and forskolin. Both the morphological examination of the cells (FIG. 11A) and the quantitative analysis (FIG. 11B) following TUNNEL assay showed that there were more apoptotic cells in cells infected with the bacteria and treated with either inhibitors. Overall, the results thus obtained demonstrated that gastric epithelial cells infected with H. pylori were sensitive to both inhibitors of the hedgehog signaling pathway.

### EXAMPLE 16

Inhibition of Hedgehog Signaling Pathway in PLC/PRF/5 Cells by Reducing Expression of Hepatitis Virus

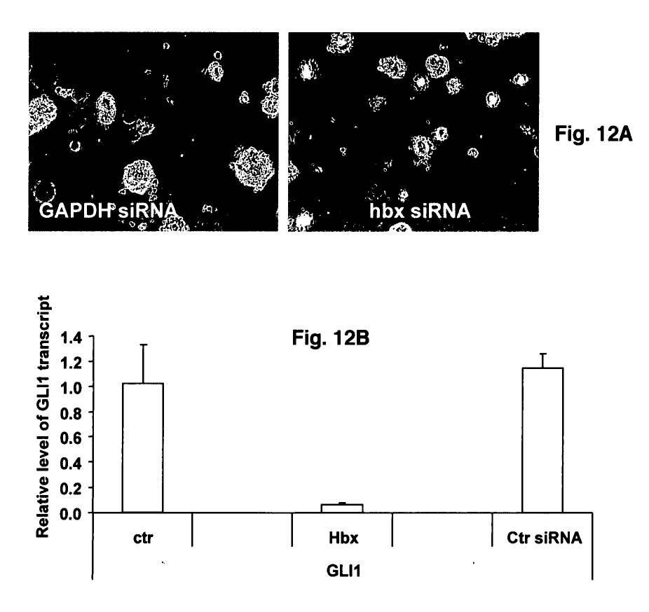

It is known that PLC/PRF/5 cells contain active hepatitis virus B replication. The present invention demonstrates that inhibition of HBV replication by knocking down hepatitis B virus X protein (hbx) caused reduced cell growth in soft agar (FIG. 12A). This indicated that HBV-mediated cell growth of PLC/PRF cells was associated with hedgehog signaling. To prove this, HBV activity was reduced by knocking down hbx using specific siRNA and examining the expression of Gli1. It was predicted that the hbx siRNA would reduce the expression of GLI1. The present invention demonstrated that hbx siRNA treatment of PLC/PRF/5 cells resulted in 90% reduction of GLI1 transcript (FIG. 12B), thereby suggesting that HBV infection regulated hedgehog signaling.

The following references were cited herein:
Athar et al., 2004, Cancer Res. 64, 7545-7552.
Chi et al., 2006, Cancer Lett. Epub Jan. 27, 2006.
Ma et al., 2006, Int J cancer 118, 139-148.
Ma et al., 2005, Carcinogensis 26, 1698-1705.
Scholle, F. et al., 2004, J. Virol. 78(3): 1513-1524.
Sheng, T et al., 2004, Mol Cancer. 3: 29
Xie et al., 2001, Proc Natl Acad Sci USA 98, 9255-9259.

Any patents or publications mentioned in this specification are indicative of the levels of those skilled in the art to which the invention pertains. Further, these patents and publications are incorporated by reference herein to the same extent as if each individual publication was specifically and individually indicated to be incorporated by reference.

What is claimed is:

1. A method of preventing or treating an infection caused by a virus or a bacteria in an individual, comprising:
administering a pharmacologically effective amount of an inhibitor that inhibits a gene encoding a protein or a protein that enables replication of said virus or said bacteria, a gene encoding a protein of hedgehog signaling pathway, the hedgehog signaling pathway protein or combination thereof, such that the administration causes apoptosis, inhibits proliferation of a virally or a bacterially infected cell or a combination thereof, thereby preventing or treating the infection caused by said virus or said bacteria in the individual.

2. The method of claim 1, wherein said inhibitor inhibits Sonic hedgehog, Glioma associated oncogene-1 (Gli1) or Patch 1 (PTCH1) gene or the protein encoded by said gene.

3. The method of claim 2, wherein said inhibitor is cyclopamine, Forskolin and IBMX, siRNA for Glioma associated oncogene-1 and Glioma associated oncogene-2, Hedgehog neutralizing antibodies or recombinant hedgehog interacting protein.

4. The method of claim 1, wherein said virus causes chronic viral infection, wherein said virus hepatitis C virus, hepatitis B virus, Human papillomavirus, Epstein-Barr virus or Herpes simplex virus.

5. The method of claim 1, wherein said bacteria causes a chronic bacterial infection, wherein said bacteria is H. pylori, Bartonellae or Agrobacterium.

6. The method of claim 1, wherein said administration treats a precancerous condition or cancer in the individual.

7. The method of claim 6, wherein the cancer is selected from the group consisting of liver cancer, stomach cancer, cervical cancer, lymphoma, oral squamous cell carcinoma or a vascular tumor.

8. A method of preventing or treating a cancer caused by activated hedgehog signaling pathway in an individual, comprising:
administering a pharmacologically effective amount of an inhibitor that inhibits viral or bacterial replication and/or inhibits activation of the hedgehog signaling pathway, thereby preventing or treating the cancer caused by the activated hedgehog signaling pathway in the individual.

9. The method of claim 8, wherein said inhibition causes apoptosis and/or inhibits proliferation of virally or bacterially infected cell, causes apoptosis and/or inhibits proliferation of a cancer causing cell or a combination thereof.

10. The method of claim 8, wherein the activation of hedgehog signaling pathway is inhibited by inhibiting a gene encoding a protein of the hedgehog signaling pathway or a hedgehog signaling pathway protein.

11. The method of claim 10, wherein said inhibition inhibits Sonic hedgehog, Glioma-associated oncogene-1, Patch1 gene or protein encoded by the gene.

12. The method of claim 11, wherein said inhibitor is cyclopamine, Forskolin and IBMX, siRNA for Glioma associated oncogene-1 and Glioma associated oncogene-2, hedgehog neutralizing antibodies or recombinant hedgehog interacting protein.

13. The method of claim 8, wherein said cancer is caused due to a chronic viral or a chronic bacterial infection.

14. The method of claim 13, wherein said chronic viral infection is caused by hepatitis C virus, hepatitis B virus, Human papillomavirus, Epstein-Barr virus or Herpes simplex virus.

15. The method of claim 14, wherein said chronic bacterial infection is caused by H. pylori, Bartonellae, or Agrobacterium.

16. The method of claim 8, wherein the individual has cancer or is diagnosed with a precancerous condition leading to said cancer, wherein the cancer is liver cancer, stomach cancer, cervical cancer, lymphoma oral squamous cell carcinoma or vascular tumor.

17. A method of diagnosing an individual with a cancer associated with hedgehog signaling pathway, comprising:
obtaining biological sample from the individual; and
determining expression of proteins associated with the hedgehog signaling pathway, wherein the detection of the protein in the sample indicates that the individual has the cancer associated with the hedgehog signaling pathway.

18. The method of claim 17, further comprising the step of concomitantly measuring the level of beta-catenin in said sample.

19. The method of claim 17, wherein said protein is Shh, PTCH1 or Glioma associated oncogene-1.

20. The method of claim 17, wherein the biological sample is a blood sample or a fine needle aspiration specimen.

21. The method of claim 17, wherein said individual is likely to suffer from or suspected of having a cancer or a disease caused by activated hedgehog signaling pathway.

22. The method of claim 21, wherein the disease is caused by a chronic viral or a chronic bacterial infection.

23. The method of claim 22, wherein the disease caused by the chronic viral infection is hepatitis, cervical inflammation, or periodontitis.

24. The method of claim 22, wherein the disease caused by the chronic bacterial infection is stomach ulcers, gastritis, lymphadenitis or vertebral osteomyelitis.

25. The method of claim 21, wherein the cancer is liver cancer, stomach cancer, cervical cancer, lymphoma, oral squamous cell carcinoma or vascular tumor.
